(19) United States
(12) Patent Application Publication
(10) Pub. No.: US 2023/0110419 A1
(43) Pub. Date: Apr. 13, 2023

(54) PRODUCT RECOMMENDATION METHOD

(71) Applicant: BEIERSDORF AG, Hamburg (DE)

(72) Inventors: Sven CLEMANN, Hamburg (DE); Soeren JASPERS, Schenefeld (DE); Maria del Pilar BONILLA-TOBAR, Berlin (DE); Michael SIEVERS, Hamburg (DE); Sonja PAGEL-WOLFF, Halstenbek (DE); Juan Manuel GRADOS-LUYANDO, Hamburg (DE); Katarzyna ZIOLKOWSKA, Berlin (DE)

(21) Appl. No.: 17/905,663

(22) PCT Filed: Feb. 3, 2021
(86) PCT No.: PCT/EP2021/052561

§ 371 Date: Sep. 6, 2022

(30) Foreign Application Priority Data
Mar. 12, 2020 (DE) 10 2020 203 189.4

(51) Int. Cl. A45D 44/00; G06T 7/10

(52) U.S. Cl. CPC A45D 44/005; G06T 7/10; A45D 2044/007; G06T 2207/10064; G06T 2207/30088

(57) ABSTRACT

The invention relates to a method for recommending cosmetics, skin care products and/or dermatological products that are adapted to the condition of the skin.

The invention relates to a method for recommending cosmetics, skin care products and/or dermatological products tailored to the condition of the skin.

There are many methods for recommending skin care products. Generally, the current condition of the skin of an individual is determined in a computer-assisted manner by measurement of at least one relevant skin parameter. The condition of the skin is used as a basis for selecting the optimal skin care product for the determined condition of the skin from a plurality of possible skin care products and suggesting it to the individual for use.

In the context of the invention, “skin care product” should be understood to mean all preparations and articles that can be applied to the skin and, once applied, influence the condition of the skin as a whole or at least one skin parameter but do not thereby unduly damage the skin. “Skin care product” should in particular be understood to mean preparations such as cosmetic preparations, dermatological preparations and/or preparations for coloring of the skin, and articles that are placed onto the skin or attached thereto, especially (face) masks or patches. Very particularly preferably, “skin care product” should be understood to mean cosmetic preparations and face masks in the context of the invention.

Suitable skin parameters to be measured are in particular the skin moisture, which is usually determined by means of impedance or capacitance measurement.

In the wake of growing technical possibilities, it has become possible to determine further, more complex skin parameters, such as roughness/wrinkle depth, flakiness, color variations, number and size of skin blemishes (e.g., acne), and skin spots (for example due to redness, liver spots or pigmentation spots).

A good example of the complex determination of the skin color can be found in U.S. Pat. No. 10,321,748, the color analysis in this case leading to the custom production of a cosmetic preparation that is adapted to the predominant skin color (that is to say a custom-produced makeup preparation).

Other skin parameters can also be used to recommend or specifically produce a preparation that suits the condition of the skin and improves the condition of the skin.

It is for instance conceivable to use the skin roughness/wrinkle depth as a basis for giving precise recommendations for active-ingredient-containing skin care products in order to reduce the skin roughness/wrinkle depth. It would likewise be possible, in the case of irregular color distribution of the skin, such as reddened irritations or pigmentation spots, to recommend appropriate products having active ingredients for equalization (lightening or increase in pigmentation) of the skin color.

However, the disadvantage of all known methods is that the condition of the skin determined solely from skin parameters is subject to large errors. By way of example, ambient temperature and/or atmospheric humidity have a major influence on blood flow (skin reddening) and on skin shine (reflection behavior of the skin). Stress factors such as physical and/or emotional stress and/or environmental influences also manifest themselves in the form of deviations of the condition of the skin from the normal condition of the skin without these stresses.

Especially the inclusion of environmental influences such as air pollution, weather (solar radiation or rain) and time of day is important for determining the base condition of the skin.

In the context of the invention, the base condition of the skin is the condition of the skin that prevails without any particular external and internal influences.

For a person skilled in the art, it was not foreseeable that a method as claimed in claim 1 remedies the disadvantages of the known processes and methods.

The invention therefore relates to a method for product recommendation, characterized in that the following method steps are performed:

1a) recording one or more images of at least one area of skin of an individual (test subject) and

1b) determining the skin moisture in the region of the recorded area of skin and

1c) determining the geo-coordinates at which the measurement has been performed,

2) transmitting the image information and skin moisture and geo-coordinates (collectively the basic data A or dataset A) to a computer infrastructure, wherein the computer infrastructure has at least one data store and one processor, wherein the processor is connected to the data store, and wherein the data store contains the inventory data in addition to further data, wherein the inventory data consist of skin profiles C that are assigned to one or more skin care products and classifications,

3) linking the basic data A to further information (extrinsic information) to form the dataset B (test subject data),

4) calculating the current skin profile D of the individual by evaluation of the dataset B,

5) comparing the current skin profile D with skin profiles C in the inventory data,

6) outputting the best matches between current skin profile D and skin profiles C in the inventory data, stating the skin care products and optionally one or more classifications assigned to the skin profiles C.

Method steps 1a to 1c are carried out on a test subject. The test subject is generally also responsible for the start of the transmission (method step 2).

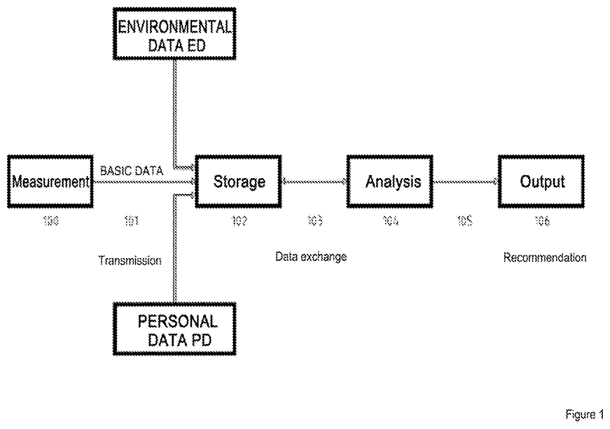

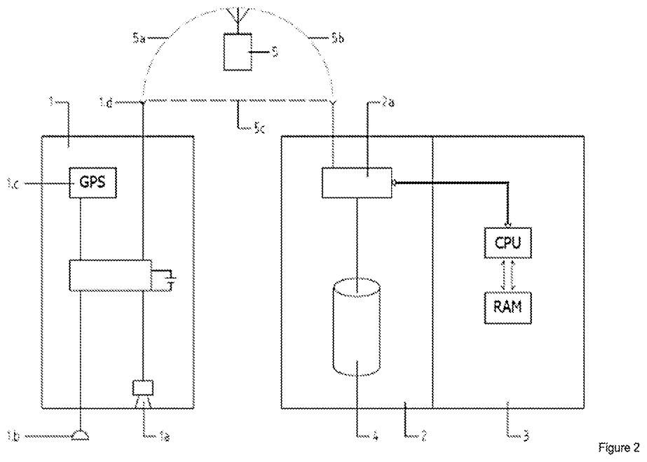

FIGS. 1 to 2 show some diagrams and flowcharts that support the description of the inventive method.

The figures represent only exemplary and preferred embodiments of the method and are not intended to limit the fundamental configuration and performance of the method.

FIG. 1 schematically represents the main steps of the method. After the measurement with a measuring arrangement (100), the measurement data are transmitted (101) into a data storage device (102) having at least one data store. For the computer-assisted analysis of the stored data, data are alternately exchanged (103) between the data storage device (102) and a computer-assisted analysis unit (104). The analysis comprises the analysis of the dataset in order to determine the skin profile D and the subsequent comparison of the skin profile D with the skin profiles C present as inventory data. The result of the analysis is transmitted (105) by the computer-assisted analysis unit (104) to an output unit (106). The result presented on the output unit (106) primarily represents a product recommendation. In addition, further information relating to the condition of the skin can also be output on the output unit.

Measurement 100

In a preferred embodiment (FIG. 2), the steps of recording one or more images with a camera (1a), determining the skin moisture with a moisture sensor (1b) and determining the geo-coordinates via a GPS receiver (1c) are carried out using a handheld device (1) which corresponds to the measuring arrangement (FIG. 1/100). All steps of recording and determining as a whole are referred to as measurement in the context of the invention.

In a particularly preferred embodiment of the handheld device, the ambient temperature and/or the atmospheric humidity in the surroundings of the handheld device are also measured and recorded in addition to the skin moisture and the image recording.

It is advantageous to record at least two images of different camera settings of the same area of skin (referred to below as image series). Preferred here is the combination of a recording of the skin surface (white light recording) in conjunction with a cross-polarized recording of the skin surface or the combination of a recording of the skin surface (white light recording) in conjunction with a fluorescence recording of the skin surface.

Insights and measurement results from the upper skin layers within the skin can be derived from the cross-polarized recording, since this looks so to speak below the uppermost skin layer as the light is not reflected at the skin surface.

Blue light makes it possible to excite specific protein structures on the skin surface and to determine the number thereof from the emitted light. This is a sign that bacteria have colonized the skin surface. The excitation is preferably effected at a wavelength in the range of 400 to 420 nm. It is an advantage of these fluorescence recordings that no filter is needed.

It is particularly preferred to record an image series that comprises a normal image (white light recording), a cross-polarized image and a fluorescence image of an area of skin.

Transmission 101

Subsequently, the collected data and images are transmitted to a data storage device (2) that can store and process the data. The transmission (5c) is preferably effected wirelessly, for example by way of NFC, WiFi or Bluetooth communication. The handheld device (1) has a transmission device (1d) for this purpose.

The data store (4) is connected to a receiving device (2a) either directly or via network devices.

The data collected by the measuring arrangement are referred to as basic data A in the context of the invention.

It is also within the context of the inventive method to first transmit (transmission path 5a) the data and images to a mobile device (5), for example a smartphone, and to then transmit (transmission path 5b) the data and images from the mobile device to the data storage device (2). This is referred to as indirect transmission path in the context of the invention. It is an advantage of this arrangement that all transmission paths of the mobile device can be used, including for example GPRS, UMTS and LTE.

It is also possible in this alternative transmission mode for skin moisture data and images to be linked to the geo-coordinates only on the mobile device. The handheld device then does not need its own GPS receiver.

In a preferred embodiment, the data store is a database, for example SQL database, in which the datasets are stored in a structured manner.

Storage 102

As soon as the data and images have been transmitted to the data storage device, the data are analyzed in a computer-assisted analysis device (3, FIG. 1/104), especially a computer.

A quality control of the basic data is performed before the data analysis. During the inspection, it can for example be checked whether the images show an uncovered area of skin, whether the images have sufficient sharpness, from which area of skin the image originates or whether the images of an image series show the same area of skin.

Yet further features (extrinsic information) can be added to the dataset of the basic data. Especially non-personal data such as the location of the measurement obtained from the GPS information and the weather conditions prevailing during the measurement. Environmental data such as pollen count and pollution level can also be transmitted (taken from other databases and systems) by querying appropriate services and servers. These additional data as a whole are referred to as environmental data in connection with the invention. Especially the weather conditions at the location and at the time of the measurement have an influence on the condition of the skin and should be taken into account in the evaluation.

To assess the basic data and any associated environmental data, it is advantageous to link further, person-related data to the basic data. These include in particular the age of the test subject, their ethnic origin, illnesses—such as diabetes or high blood pressure—, medication taken, alcohol consumption, drug consumption, sun exposure before the measurement, pulse rate, weight, eating habits, hormonal status but also biomarkers determined in particularly detailed surveys, for example genetic, epigenetic or else microbial biomarkers. In addition there are data about regularly used care products or treatments. These person-related data are referred to as personal data in the context of the invention.

Personal data are preferably collected by way of a mobile device which is connected to the measuring device. In the context of the above-described indirect transmission path of the basic data, the person-related data can be delivered to the data storage device together with the basic data.

Basic data, environmental data and personal data form the extended dataset for a test subject and are referred to as test subject data (dataset B) in the context of the invention.

Analysis 104

The test subject data stored in the data storage device are analyzed by way of a computer-assisted analysis unit (3). The computer-assisted analysis unit is preferably a computer that has at least one processor and corresponding memory and is capable of being able to process data by means of programmable computing rules. The data storage device and computer-supported analysis unit are in communication with one another, so that the computer-assisted analysis unit can retrieve data from the data storage device and can send data to the data storage device. The data storage device and computer-assisted analysis device may form one technical unit or else exist separately from one another (for example cloud solution for the data storage and local (large) computing system or workstation for the evaluation).

The images are evaluated in the analysis unit either separately or in a combination with each other in order to be able to determine different parameters that describe the condition of the skin. The recorded images and their variations are also used in order to further refine (train) these evaluation algorithms or to develop new algorithms with the aid of artificial intelligence methods.

Each measurement preferably yields information about the condition of the skin and a suggestion of products to be used and/or recommendations for measures in order to maintain and/or to improve the condition of the skin. Recommendations for measures and/or products that minimize or prevent a skin problem (preventive recommendation) are also advantageous.

1.-11. (canceled)

12. A method for product recommendation, wherein the method comprises:
1a) recording one or more images of at least one area of skin of an individual (test subject) and
1b) determining skin moisture in a region of the recorded area of skin and
1c) determining geo-coordinates at which a measurement has been performed,
2) transmitting image information and skin moisture and geo-coordinates (collectively “the basic data A”) to a computer infrastructure comprising at least one data store and one processor, the data processor being connected to the data store, and the data store containing inventory data in addition to further data, wherein the inventory data consist of skin profiles C that are assigned to one or more skin care products and classifications,
3) linking the basic data A to extrinsic information to form dataset B,
4) calculating a current skin profile D of the individual by evaluation of the dataset B,
5) comparing the current skin profile D with skin profiles C in the inventory data,
6) outputting the best matches between current skin profile D and skin profiles C in the inventory data, stating the skin care products and optionally one or more classifications assigned to the skin profiles C,
and wherein 1a and/or 1b and/or 1c can be carried out simultaneously or successively.

13. The method of claim 12, wherein in 1a two images of different camera settings of the same area of skin are taken.

14. The method of claim 12, wherein the images taken in 1a are selected from white light recording, cross-polarized recording and/or fluorescence recording.

15. The method of claim 12, wherein in 1a at least one white light recording and at least one fluorescence recording is taken.

16. The method of claim 12, wherein in 1a at least one white light recording, one cross-polarized image and one fluorescence image of an area of skin is taken.

17. The method of claim 12, wherein the extrinsic information comprises ambient temperature and/or atmospheric humidity in the surroundings.

18. The method of claim 12, wherein at least 1a and 1b are performed using a hand-holdable device that is brought into contact with a skin surface of the individual.

19. The method of claim 18, wherein the hand-holdable device comprises a transmission device.

20. The method of claim 12, wherein in 2, information is transmitted wirelessly.

21. The method of claim 20, wherein information is transmitted by at least one of NFC, WiFi or Bluetooth communication.

22. The method of claim 12, wherein the external information comprises personal data.

23. The method of claim 22, wherein the personal data are collected by a mobile device.
